(19) United States
(12) Patent Application Publication
(10) Pub. No.: US 2023/0301524 A1
(43) Pub. Date: Sep. 28, 2023

(54) MONITORING ASSISTANCE SYSTEM AND MONITORING ASSISTANCE METHOD

(71) Applicants: YUTAKA ELECTRONICS CO., LTD., Hashima-shi, Gifu (JP); GREED LTD., Gifu-shi, Gifu (JP)

(72) Inventors: Yutaka AONO, Hashima-shi (JP); Futoshi IMAMICHI, Gifu-shi (JP)

(73) Assignees: YUTAKA ELECTRONICS CO., LTD., Hashima-shi, Gifu (JP); GREED LTD., Gifu-shi, Gifu (JP)

(21) Appl. No.: 18/022,160

(22) PCT Filed: Aug. 25, 2021
(86) PCT No.: PCT/JP2021/031225

§ 371 Date: Feb. 19, 2023

(30) Foreign Application Priority Data
Aug. 27, 2020 (WO) PCT/JP2020/032359

(51) Int. Cl. A61B 5/0205; A61B 5/00

(52) U.S. Cl. CPC A61B 5/02055; A61B 5/0002; A61B 5/681; A61B 5/6844; A61B 5/746; A61B 5/14542; A61B 2562/08

(57) ABSTRACT

In a monitoring assistance system that includes a wearable terminal which is worn by a person to be monitored and which continuously obtains vital data, a watcher terminal associated with the wearable terminal, and a management server communicably connected to the watcher terminal and/or the wearable terminal and that processes vital data to which an identification code of the wearable terminal is attached, the vital data includes pulse rate data and blood oxygen level data, it is determined, in a case where the pulse rate data and the blood oxygen level data is not being detected, that a non-wearing state, where the wearable terminal is removed from the person, has been established, and it is determined, in a case where the pulse rate data is not being detected and the blood oxygen level data is being detected, that a pulse of the person to be monitored has stopped.

### BACKGROUND OF THE INVENTION

### Field of the Invention

The present invention relates to a monitoring assistance system and a monitoring assistance method.

### Description of the Related Art

Even if there are signs before onset of disease or a sudden change in a medical condition, a person is often unaware of these signs. This is especially true for the elderly, as their physical senses are dull. Even if a person feels that he/she is not in good physical condition, it is often difficult for the person to properly communicate to a medical care worker what is wrong with him/her. Especially in the case of a patient with dementia, even a medical interview is difficult where the patient is asked about his/her physical condition and symptoms. In addition, although it is recommended that blood pressure and body temperature be taken at regular times each day to monitor one’s own physical condition and maintain health, many people are unable to continue to do so. A person’s inability to “notice”, “communicate”, or “continue” might thus delay detection of physical abnormalities and make diseases more serious.

Wearable terminals that can be worn on a wrist like wristwatches to measure a pulse rate, body temperature, and the like non-invasively without attaching electrodes to a human body have been proposed (e.g., PTL 1). Such wearable terminals are utilized, for example, by health-conscious people for their own health management. During sports, such people adjust a load of exercise on the basis of changes in a pulse rate or grasp the amount of exercise on the basis of the number of steps taken.

The present inventors thought that such wearable terminals could be used to detect physical abnormalities at early stages by having elderly or sick people wear the wearable terminals. Conventional wristwatch-type wearable terminals are designed for temporary use, such as during sports, and elaborate designs thereof regarding monitor indications result in large power consumption. Because a battery needs to be frequently charged, such wearable terminals cannot be continuously used. The present inventors, on the other hand, have suppressed power consumption by devising a data measurement method and an output format, thereby making it possible to use such wearable terminals continuously for a long period of at least one week without charging a battery. This makes it possible to continuously obtain data (vital data) regarding a body of a person who wears the wearable terminal. With a conventional wearable terminal, however, a pulse rate, body temperature, and the like are only displayed on a monitor of the wearable terminal. It is therefore necessary for a person to consciously read values and monitor changes in the values, and it is difficult for elderly or sick people to perform such tasks.

The present applicants, therefore, have proposed a monitoring assistance system and a monitoring assistance method capable of detecting physical abnormalities at early stages by obtaining vital data regarding a person who wears a wearable terminal without requiring the person to perform any tasks (the application pertaining to this proposal does not fall under a category of publicly known literature because the application has not yet been published). In the proposed monitoring assistance method, vital data including pulse rate data is continuously obtained for a person who wears a wearable terminal, stored within the monitoring assistance system as a database, and analyzed to detect occurrence of abnormalities and signs of abnormalities.

It is desired to detect death of a wearer as one of the abnormalities that occur. It can be thought that death of a wearer (a pulse stop) can be detected on the basis of a fact that a wearable terminal no longer detects a pulse rate. Because the pulse rate is not detected either even in a non-wearing state, where the wearer is not wearing the wearable terminal for bathing or other purposes, however, it is difficult to distinguish whether a cause of an undetected pulse rate is a pulse stop or the non-wearing state. In the case of a conventional sport-type wearable terminal, a health-conscious person uses the wearable terminal to monitor his/her own vital data, and no problem arises because when the person has removed the wearable terminal, the person is aware that he/she is not wearing the wearable terminal. When occurrence of abnormalities and signs of abnormalities are to be detected by continuously obtaining vital data regarding a person to be monitored, such as an elderly person or a sick person, on the other hand, it is very important to distinguish whether a cause of an undetected pulse rate is a pulse stop or the non-wearing state.

### CITATION LIST

### Patent Document

Patent Document 1: Japanese Patent Application Laid-open No. 2017-18236

### SUMMARY OF THE INVENTION

In view of the above circumstances, therefore, the present invention aims to provide a monitoring assistance system and a monitoring assistance method capable of distinguishing, when vital data including pulse rate data is to be continuously obtained for a wearer of a wearable terminal but the pulse rate data is not being detected, whether a cause of the undetected pulse rate data is a pulse stop or a non-wearing state, where the wearable terminal is removed.

In order to solve the above problem, a monitoring assistance system in the present invention includes
“a wearable terminal that is worn by a person to be monitored and that continuously obtains vital data;
a watcher terminal associated with the wearable terminal; and
a management server communicably connected to at least one of the watcher terminal or the wearable terminal,
in which at least one of the watcher terminal or the management server includes first vital data processing means for receiving the vital data to which an identification code of the wearable terminal is attached and performing data processing on the vital data,
in which the vital data includes pulse rate data and blood oxygen level data, and
in which the first vital data processing means determines, in a case where the pulse rate data is not being detected and the blood oxygen level data is not being detected, that a non-wearing state, where the wearable terminal is removed from the person to be monitored, has been established, and, in a case where the pulse rate data is not being detected and the blood oxygen level data is being detected, that a pulse of the person to be monitored has stopped, generates an alert signal, and causes the watcher terminal to notify”.

In the monitoring assistance system, a wearable terminal worn by a person to be monitored continuously obtains vital data including data regarding a pulse. As described in detail later, physical events including disease onset and physical condition changes, such onset of atrial fibrillation, awakening during sleep, fever, a sign of a pulse stop, and a pulse stop, manifest as changes in the vital data. In the monitoring assistance system, first vital data processing means, which is a functional component of the system, detects physical events by detecting changes in the vital data obtained continuously.

As described above, it can be considered that a pulse stop, which is one of physical events, can be detected on the basis of a fact that pulse rate data is zero (not detected) in the vital data. A pulse rate is not detected either, however, when a person to be monitored has removed the wearable terminal. When the pulse rate data is not being detected, therefore, it is necessary to distinguish whether a cause of the undetected pulse rate data is a pulse stop or the non-wearing state, where the wearable terminal is removed.

In a case where the pulse rate data is not being detected and the blood oxygen level is not being detected, therefore, the first vital data processing means determines that the non-wearing state has been established. In a case where the pulse rate data is not being detected but the blood oxygen level data is being detected, on the other hand, the first vital data processing means determines that a pulse has stopped, generates an alert signal, and transmits the alert signal to the watcher terminal. This is because the inventors of the present invention paid attention to the fact that even if a pulse stops, the blood oxygen level continues to be detected insofar as the wearable terminal is being worn.

With the monitoring assistance system, therefore, when vital data including pulse rate data is to be obtained for a wearer of the wearable terminal but the pulse rate data is not being obtained, whether a cause of the undetected pulse rate data is a pulse stop or the non-wearing state, where the wearable terminal is removed, can be clearly distinguished using not only the pulse rate data but also the blood oxygen level data.

In addition to the above configuration of the monitoring assistance system in the present invention,
the vital data may include body temperature data,
the body temperature data may be deep body temperature data calculated on a basis of body surface temperature and infrared radiation temperature measured by the wearable terminal, and
in a case where the pulse rate data is not being detected and the blood oxygen level data and the body temperature data are being detected, the first vital data processing means may determine that the pulse of the person to be monitored has stopped.

With this configuration, body temperature data is included as vital data and used to detect occurrence of abnormalities and signs of abnormalities, as well as to determine a pulse stop. Deep body temperature data calculated on the basis of body surface temperature and infrared radiation temperature measured by the wearable terminal is used as body temperature data. The medically recognized “body temperature” is deep body temperature measured at an armpit, under a tongue, or in an anus, but cannot be directly measured by the wearable terminal. Therefore, it can be conceivable to use body surface temperature, which can be easily measured by the wearable terminal, as body temperature data, but in this case, the sensor for detecting the body surface temperature undesirably measures air temperature when the wearable terminal is not being worn. When the air temperature is close to the body surface temperature, such as in summer, therefore, it is difficult to distinguish whether the body surface temperature or the air temperature is being measured.

In this configuration, on the other hand, body surface temperature and infrared radiation temperature are combined to calculate body temperature data. When the wearable terminal is not being worn, the sensor for detecting body surface temperature undesirably measures air temperature, but a sensor for detecting infrared radiation temperature does not detect temperature. Body temperature data, therefore, is not detected in the non-wearing state. Because body temperature decreases after death by about 1° C. every 10 hours in an ordinary environment, the body temperature hardly decreases for a while after a pulse stops and can be detected. The body temperature data, therefore, can be used to determine a pulse stop.

In addition, infrared radiation temperature is not easily affected by air temperature and reflects temperature in a deep part of a body affected by blood flow, but accuracy in converting a detected value of infrared radiation into a temperature is undesirably low. By combining two sensing methods, namely body surface temperature and infrared radiation temperature, however, it is possible to accurately calculate body temperature data close to an actually measured value of deep body temperature, as described in detail later.

Next, a monitoring assistance method in the present invention is “the method used in a monitoring assistance system including
a wearable terminal that is worn by a person to be monitored and that continuously obtains vital data,
a watcher terminal associated with the wearable terminal, and
a management server communicably connected to at least one of the watcher terminal or the wearable terminal,
in which the vital data to which an identification code of the wearable terminal is attached is processed, the monitoring assistance method including:
   including pulse rate data and blood oxygen level data in the vital data;
   determining, in a case where the pulse rate data is not being detected and the blood oxygen level data is not being detected, that a non-wearing state, where the wearable terminal is removed from the person to be monitored, has been established; and
   determining, in a case where the pulse rate data is not being detected and the blood oxygen level data is being detected, that a pulse of the person to be monitored has stopped”.

This is the monitoring assistance method used in the monitoring assistance system described above. The determination as to the non-wearing state may be a process performed by the first vital data means in at least one of the watcher terminal or the management server, or may be a process performed by the wearable terminal itself. In the latter case, the wearable terminal that has determined that the non-wearing state has been established may transmit a signal indicating the non-wearing state to the first vital data processing means. If at least one of pulse rate data or blood oxygen level data is then detected, it may be determined that the wearable terminal is being worn again and a signal indicating that the wearable terminal is being worn again may be transmitted to the first vital data processing means.

As described above, according to the present invention, a monitoring assistance system and a monitoring assistance method capable of distinguishing, when vital data including pulse rate data is to be continuously obtained for a wearer of a wearable terminal but the pulse rate data is not being obtained, whether a cause of the undetected pulse rate data is a pulse stop or a non-wearing state, where the wearable terminal is removed.

### BRIEF DESCRIPTION OF THE DRAWINGS

FIG. 1 is a diagram illustrating the configuration of a monitoring assistance system according to an embodiment of the present invention.

FIG. 2A illustrates monitor indications on a wearable terminal in a non-wearing state, and FIG. 2B illustrates monitor indications on the wearable terminal after a wearer’s pulse stops.

FIG. 3 is a graph showing temporal changes in a pulse rate at a time of an awakening at night.

FIG. 4A is a graph showing temporal changes in a pulse rate of a healthy person at rest, and FIG. 4B is a graph showing temporal changes in a pulse rate of a patient with atrial fibrillation at rest.

FIG. 5A is a graph showing temporal changes in a pulse rate and body temperature of a bedridden person who is not given life-support measures one to two weeks before a pulse stops, and FIG. 5B is a graph showing temporal changes of the pulse rate and the body temperature of the same person on a day when death was declared.

### DESCRIPTION OF THE EMBODIMENTS

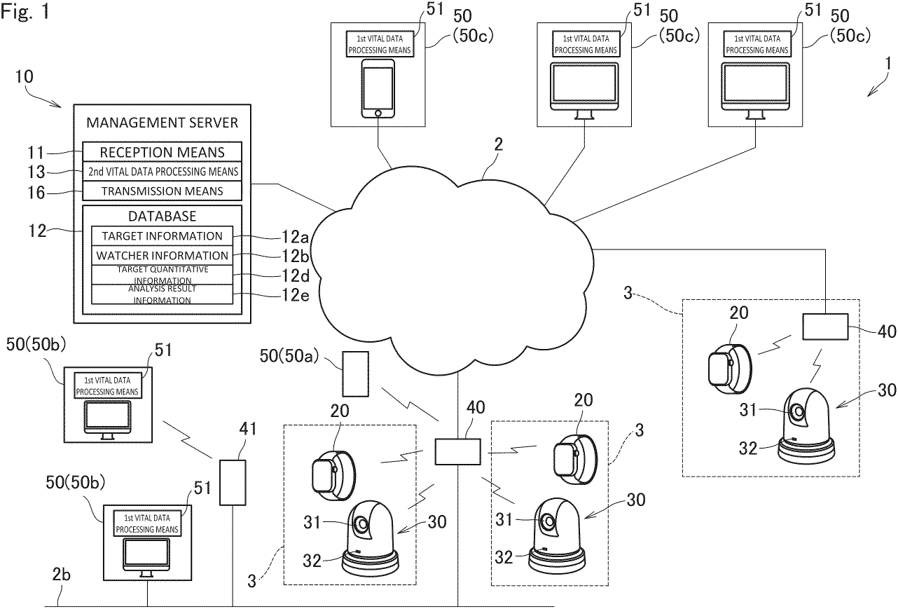

A monitoring assistance system 1 according to a specific embodiment of the present invention will be described hereinafter with reference to the drawings. Persons to be monitored by the monitoring assistance system 1 according to the present embodiment include residents of nursing homes, patients in hospitals, and persons who live at home but need medical care or nursing care. The monitoring assistance system 1 according to the present embodiment includes, as FIG. 1 illustrates a schematic configuration thereof, a management server 10, wearable terminals 20, camera apparatuses 30, dedicated transceivers 40, and watcher terminals 50.

The wearable terminal 20 is a terminal worn on a wrist of a person to be monitored like a wristwatch and includes a CPU, a storage device, and a monitor. The wearable terminal 20 obtains data regarding a pulse, skin temperature, the number of steps taken (activity level), blood pressure, and SpO₂ (oxygen saturation level) as vital data regarding the persons. The data is obtained continuously at short intervals of one to two minutes.

### Measurement of Data Regarding a Pulse

The data regarding a pulse is a pulse rate and PPIs and measured by photoelectric pulse wave measuring method. Photoelectric pulse wave measuring method is roughly divided into transmission pulse wave measurement, in which the amount of change in light transmitted through a body is measured, and reflection pulse wave measurement, in which the amount of change in light reflected within a body is measured. In the present embodiment, reflection pulse wave measurement using a reflection pulse wave sensor is employed. More specifically, when volume of blood vessels changes due to pulsation, the amount of hemoglobin in blood also changes. Since hemoglobin absorbs green light, pulsation, and thus a pulse wave, can be detected by utilizing a fact that reflected light radiated by green LEDs onto the blood vessels varies depending on the amount of hemoglobin. The PPIs are peak intervals (in seconds) of a pulse waveform, and the pulse rate is an average PPI per minute (60 divided by an average PPI). In the present embodiment, HRV (heart rate variability) is also measured as data regarding a pulse in addition to the pulse rate and the PPIs.

### Measurement of Body Temperature

Body temperature data indicates a result of measurement by a sensor converted into deep body temperature through correction. The deep body temperature is medically recognized as “body temperature” but cannot be directly measured by a wearable terminal because the deep body temperature should be measured at an armpit, under a tongue, or in an anus. In the present embodiment, therefore, the deep body temperature is obtained on the basis of a measured value of body surface temperature and a measured value of infrared radiation temperature. Here, the body surface temperature is measured by detecting, using a thermistor, a change in electrical resistance caused by a temperature change. In measurement of the infrared radiation temperature, infrared radiation emitted by every object is detected and converted into a temperature of the object. In the case of a living body, the deep body temperature, which is affected by blood flow, is reflected.

For the conversion into the deep body temperature, a relationship between measured values of body surface temperature and infrared radiation temperature and an actually measured value of deep body temperature is investigated in advance, and a correction formula or a correction database determined on the basis of the relationship is used. Even if a relationship between body surface temperature and an actually measured value of deep body temperature is investigated in order to obtain deep body temperature only on the basis of measurement of the body surface temperature, the relationship cannot be accurately obtained because the body surface temperature is affected by air temperature. In addition, since there are large individual differences in blood flow, deep body temperature, which is affected by blood flow, often differs even if the body surface temperature is the same, that is, a correlation between body surface temperature and deep body temperature is often low. Whereas infrared radiation temperature reflects deep body temperature, which is affected by blood flow, and is less affected by air temperature, accuracy in detecting emitted infrared energy with a sensor and converting the infrared energy into a temperature is low. In the present embodiment, deep body temperature can be accurately obtained by combining body surface temperature and infrared radiation temperature, each of which has its own disadvantages on its own, investigating a relationship between the combination and an actually measured value of deep body temperature, and determining a correction formula or a correction database.

Deep body temperatures (deep body temperatures obtained through correction) of Subject A (male in his 30s) and Subject B (male in his 20s) obtained on the basis of body surface temperatures and infrared radiation temperatures measured with wearable terminals were actually compared with actually measured values of deep body temperature at that time. The actually measurement of deep body temperature was performed by measuring temperature of an armpit with a mercury thermometer-type thermometer. The measurement was performed seven times at different times for each subject. Table 1 shows the values of deep body temperature (BT) obtained through correction and the measured values of body surface temperature (ST) contrasted with the actually measured values of deep body temperature (AMV).

TABLE 1

| | Subject A | | | Subject B | | |
|---|---|---|---|---|---|---|
| | AMV (°C) | BT (°C) | ST (°C) | AMV (°C) | BT (°C) | ST (°C) |
| 1 | 36.5 | 36.7 | 35.5 | 36.4 | 36.3 | 35.3 |
| 2 | 36.5 | 36.7 | 35.5 | 36.6 | 36.7 | 35.5 |
| 3 | 36.6 | 36.6 | 35.6 | 36.7 | 36.8 | 35.6 |
| 4 | 36.5 | 36.6 | 35.5 | 36.5 | 36.7 | 35.3 |
| 5 | 36.8 | 36.7 | 35.6 | 36.5 | 36.7 | 35.2 |
| 6 | 36.6 | 36.6 | 35.6 | 36.6 | 36.7 | 35.4 |
| 7 | 36.7 | 36.8 | 35.6 | 36.8 | 36.8 | 35.7 |

As shown in Table 1, the measured values of body surface temperature (ST) were 1.0° C. to 1.3° C. lower than the actually measured values of deep body temperature (AMV), that is, differences were large. Differences between the deep body temperatures (BT) obtained through correction and the actually measured values of deep body temperature (AMV) were in a small range of 0° C. to ±0.2° C. Measured values of body surface temperature and infrared radiation temperature can thus be converted into a value close to an actually measured value of deep body temperature by combining and correcting the measured values.

### Measurement of Number of Steps Taken (Activity Level)

The number of steps taken (activity level) is measured by measuring, using a three-axis acceleration sensor, the number of times that three-axis acceleration has acted.

### Measurement of Blood Pressure

With respect to the blood pressure, because volume of blood vessels increases as the blood pressure increases, and accordingly the amount of hemoglobin increases, the pulse wave is detected on the basis of reflected light of light emitted from the green LEDs as in the reflection pulse wave measurement, and a blood pressure value is estimated by measuring pulse propagation from a shape of the pulse wave.

### Measurement of Blood Oxygen Level (SpO₂)

A blood oxygen level is a percentage of hemoglobin in blood bound to oxygen and a normal value thereof ranges from 96% to 100%. Hemoglobin not bound to oxygen absorbs red light well, exhibiting a dark red color. Hemoglobin bound to oxygen, on the other hand, reflects more red light (absorbance is lower), exhibiting a bright red color. Absorption and reflection of infrared light by hemoglobin, on the other hand, is not related to the oxygen level. By simultaneously radiating red light and infrared light onto blood vessels, therefore, a ratio of hemoglobin bound to oxygen and hemoglobin not bound to oxygen can be identified from a ratio of reflected light or transmitted light received by a sensor, and the blood oxygen level can be determined.

The wearable terminal according to the present embodiment simultaneously radiates red light with a wavelength of 650 nm and infrared light with a wavelength of 950 nm onto a wearer’s body, and the sensor detects reflected light of the red light and the infrared light. When a percentage of hemoglobin bound to oxygen in all hemoglobin increases, the reflected light of the red light received by the sensor increases, but the reflected light of the infrared light received by the sensor remains almost unchanged. When the percentage of hemoglobin bound to oxygen in all the hemoglobin decreases, the reflected light of the red light received by the sensor decreases, but the reflected light of the infrared light received by the sensor remains almost unchanged. The blood oxygen level, therefore, can be determined on the basis of a ratio of red light to infrared light received by the sensor.

Since changes in the blood oxygen level fluctuate (pulsate) in proportion to the amount of hemoglobin passed, the PPIs and the pulse rate can be determined on the basis of a cycle of changes in the blood oxygen level.

The wearable terminal 20 uses a battery as a power supply, but by devising a method for measuring vital data and changing information to be displayed on a monitor, the wearable terminal 20 consumes significantly less power than a conventional wristwatch-type wearable terminal, and can be used continuously for a long period of one to two weeks. Even if an elderly person who lives alone visited by a visiting medical worker only once or twice a week is the person to be monitored, therefore, it is sufficient for the visiting medical worker to replace the battery during the visit. Vital data can be continuously obtained without requiring the person himself/herself to replace the battery. A battery level may be transmitted to a watcher terminal 50 associated with the wearable terminal 20.

The camera apparatus 30 is provided in a living space 3 of the person to be monitored who wears the wearable terminal 20. The camera apparatus 30 includes a camera 31, a microphone 32, and a speaker (not illustrated). The camera apparatus 30 outputs voices transmitted from the watcher terminal 50 from the speaker, collects sounds therearound, and transmits the sounds to the watcher terminal 50. That is, the camera apparatus 30 is a remote camera through which conversation can take place. The camera apparatus 30 can turn on and off the camera 31 and the microphone 32 on the basis of signals transmitted from the watcher terminal 50 and change a shooting direction and shooting magnification of the camera 31.

The dedicated transceiver 40 is provided in the living space 3 of the person to be monitored who wears the wearable terminal 20 or a building including the living space 3. The dedicated transceiver 40 is a computer including a modem with a router function in addition to a CPU and a storage device and connected to the wearable terminal 20 and the camera apparatus 30 through wireless communication such as Wi-Fi or Bluetooth (registered trademark) and to a communication network 2 such as the Internet by wire. When persons to be monitored live in a facility such as a nursing home or a hospital, for example, the dedicated transceiver 40 may be provided in a living space 3 of each of the persons, each of floors of the facility, or for a certain number of living spaces 3.

The watcher terminal 50 is a terminal used by a watcher, who monitors the person to be monitored. The watcher may include, for example, medical care workers (hereinafter referred to as “medical workers”), visiting medical care workers such as visiting nurses and visiting care workers (hereinafter referred to as “visiting medical workers”), family members of the person. When the person to be monitored is a resident of a nursing home, for example, the watcher terminal 50 may be, for example, a terminal used by a medical worker who looks after the resident or a terminal provided for an office of the nursing home. When the person is a patient in a hospital, the watcher terminal 50 may be, for example, a terminal used by a medical worker such as a doctor or a therapist, such as a terminal used by a nurse who looks after the person or a terminal provided in a nurse station. When the person to be monitored is a person who lives at home but needs medical care or nursing care, the watcher terminal 50 may be, for example, a terminal used by a visiting medical worker who looks after the person or a terminal at an office to which the visiting medical worker belongs.

The watcher terminal 50 is a computer including a CPU, a storage device, input devices such as a keyboard and a mouse, and output devices such as a monitor and a printer. The watcher terminal 50 according to the present embodiment also includes a microphone and a speaker. The watcher’s voice can be collected by the microphone and transmitted to the camera apparatus 30, and sounds transmitted from the camera apparatus 30 can be output from the speaker. The watcher terminal 50 may be a desktop personal computer, a laptop personal computer, a tablet computer, or a smartphone.

Dedicated software for using the monitoring assistance system 1 is installed on the watcher terminal 50. As a result, the watcher terminal 50 includes reception means, transmission means (both are not illustrated), and first vital data processing means 51 as functional components. The watcher terminal 50 communicates data and signals with the dedicated transceiver 40 and the management server 10 through the reception means and the transmission means.

In the present embodiment, the watcher terminals 50 include watcher terminals 50a that wirelessly communicate with the dedicated transceivers 40 and that are connected to the communication network 2 through the dedicated transceivers 40, watcher terminals 50b that communicate with the dedicated transceivers 40 by wire over a facility communication network 2b and that are connected to the communication network 2 through the dedicated transceivers 40, and watcher terminals 50c connected to the communication network 2 without using the dedicated transceivers 40. The watcher terminals 50a and 50b, which are provided in the same facilities as the wearable terminals 20, can communicate data and signals with the wearable terminals 20 through the dedicated transceivers 40 without using the communication network 2. The watcher terminals 50a, 50b, and 50c are referred to as “watcher terminals 50” herein when the watcher terminals 50a, 50b, and 50c need not be particularly distinguished from one another.

The first vital data processing means 51 is means for immediately performing data processing on vital data obtained by the wearable terminal 20. The first vital data processing means 51 includes vital change detection means and alert means. The vital change detection means is means for detecting, on the basis of changes in the vital data, physical events including disease onset and physical condition changes of the person to be monitored. The physical events including disease onset and physical condition changes will be described in detail later.

The alert means is means for causing the watcher terminal 50 to notify of occurrence of abnormalities on the basis of detection by the vital change detection means. The notification by the watcher terminal 50 may be lighting up or blinking of a warning light, display of a warning on a monitor screen, outputting of a warning sound from the speaker, or a combination of some or all of these.

The management server 10 is a server managed by a manager of the monitoring assistance system 1 and connected to the communication network 2. The management server 10 is a computer including a CPU, a storage device, input devices such as a keyboard and a mouse, and output devices such as a monitor and a printer. The management server 10 includes reception means 11, a database 12, second vital data processing means 13, and transmission means 16 as functional components.

The management server 10 communicates, using the reception means 11 and the transmission means 16, data and signals with the watcher terminals 50a and 50b through the dedicated transceivers 40. The management server 10 also communicates, using the reception means 11 and the transmission means 16, with the watcher terminals 50c without using the dedicated transceivers 40.

The second vital data processing means 13 is means for performing data processing using target quantitative information 12d that has been accumulated for a certain period of time, the target quantitative information 12d being vital data obtained by the wearable terminals 20, associated with target information 12a, and stored in the database 12. The second vital data processing means 13 includes status information generation means, time-series pattern analysis means, and event estimation and prediction means.

The database 12 stores the target information 12a, watcher information 12b, the target quantitative information 12d, and analysis result information 12e. The target information 12a is information in which information such as gender and age (date of birth) of the persons to be monitored is associated with identification codes of the wearable terminals 20 worn by the persons. The target information 12a according to the present embodiment also includes information in which an identification code of a dedicated transceiver 40 to which each of the wearable terminals 20 transmits vital data and an identification code of a camera apparatus 30 provided in a living space 3 in which the wearable terminal 20 is provided are associated with the identification code of the wearable terminal 20. The target information 12a according to the present embodiment also includes disease information and physical condition information regarding the persons to be monitored. The disease information and the physical condition information will be described later.

The watcher information 12b is information in which the watcher terminals 50 and the wearable terminals 20 are associated with each other using identification codes thereof. The watcher information 12b also includes authentication information for the watcher terminals 50 to access the management server 10, such as IDs and passwords.

The watcher terminals 50 and the wearable terminals 20 associated with each other need not necessarily be in one-to-one correspondence. When a plurality of medical workers and visiting medical workers look after one person to be monitored, for example, a plurality of watcher terminals 50 and one wearable terminal 20 are associated with each other. When one medical worker or visiting medical worker looks after a plurality of persons to be monitored, one watcher terminal 50 and a plurality of wearable terminals 20 are associated with each other.

The target quantitative information 12d is information in which vital data obtained by the wearable terminals 20 is associated with the target information 12a. The target quantitative information 12d may also include results of data processing performed on vital data using the first vital data processing means 51. The analysis result information 12e is information in which, if a correlation is found between a time-series pattern and at least one of disease information or physical condition information as a result of an analysis conducted by the time-series pattern analysis means in the data processing performed by the second vital data processing means 13, the disease information and/or the physical condition information is associated with the time-series pattern and that is stored in the database 12.

Next, a monitoring assistance method employing the monitoring assistance system 1 according to the present embodiment will be described. First, a person to be monitored wears the wearable terminal 20. The camera apparatus 30 is installed in a living space 3 of the person, and the dedicated transceiver 40 is installed in the living space 3 or a building including the living space 3.

In the watcher terminal 50, dedicated software for using the monitoring assistance system 1 is installed and activated. The watcher terminal 50 logs in to the management server 10 by inputting authentication information over the communication network 2. As a result, the watcher terminal 50 can read the watcher information 12b, and find the wearable terminal 20 associated therewith. In addition, when the person to be monitored changes from day to day, for example, the watcher information 12b can be updated by associating, on the basis of an input from the watcher terminal 50, the watcher terminal 50 with a wearable terminal 20 worn by a new person to be monitored. When the watcher information 12b is updated, the management server 10 registers a watcher terminal 50 newly associated with a dedicated transceiver 40 associated with the new wearable terminal 20. That is, the dedicated transceiver 40 that receives vital data from the wearable terminal 20 obtains information indicating a destination of the vital data (transmission to a watcher terminal 50a, transmission to a watcher terminal 50b, or transmission to a watcher terminal 50c through the management server 10). Alternatively, the dedicated transceiver 40 may obtain, on the basis of an input from a watcher terminal 50a or 50b provided in the same facility as the wearable terminal 20 without using the management server 10, information regarding the watcher terminal 50a or 50b to which the dedicated transceiver 40 is to transmit the vital data.

When use of the monitoring assistance system 1 starts in this state, the wearable terminal 20 transmits the vital data obtained thereby with an identification code thereof attached to the vital data. When the wearable terminal 20 is associated with the watcher terminal 50a or 50b used by the watcher in the same facility such as a nursing home or a hospital, the dedicated transceiver 40 that has received the vital data from the wearable terminal 20 transmits the vital data to the watcher terminal 50a or 50b without using the communication network 2. At this time, another transceiver 41 may mediate the data transmission when the dedicated transceiver 40 that has received the vital data from the wearable terminal 20 transmits the vital data to the watcher terminal 50a or 50b.

Upon receiving the vital data, the watcher terminal 50a or 50b performs data processing using the first vital data processing means 51. In the data processing, first, the vital change detection means detects changes in the vital data and, if it is determined that the changes indicate an abnormality, the alert means generates an alert signal and transmits the alert signal to notification devices (the speaker, the warning light, and the monitor) of the watcher terminal 50a or 50b. As a result, the watcher terminal 50a or 50b notifies of occurrence of the abnormality.

The alert signal is also transmitted to the dedicated transceiver 40 that has mediated the transmission of the vital data and the camera apparatus 30 associated with the wearable terminal 20 worn by the person to be monitored in whom the abnormality has occurred. The camera apparatus 30 switches the camera 31 to a monitoring state and the microphone 32 to a sound collection state on the basis of the reception of the alert signal. As a result, the watcher who is using the watcher terminal 50a or 50b notified of the occurrence of the abnormality can immediately visually recognize, through the camera 31, a state of the person to be monitored and have a conversation by talking to the person through the speaker and listening to the person through the microphone 32. The watcher can then rush to the person in accordance with the recognized state.

When the person to be monitored and the watcher are in the same facility as described above, the vital data transmitted from the wearable terminal 20 is transmitted to the watcher terminal 50a or 50b without using the communication network 2 and subjected to the data processing. The data processing, therefore, can be performed in almost real-time and, if an abnormality occurs in the person, the watcher can respond extremely quickly.

The vital data received by the watcher terminal 50a or 50b is transmitted to the management server 10 over the communication network 2 at a certain timing along with a result of data processing performed by the first vital data processing means 51 and stored in the database 12 as target quantitative information 12d. A large amount of vital data, therefore, need not be stored in a storage device of the watcher terminal 50a or 50b.

When the person to be monitored and the watcher are geographically distant from each other, that is, when the person to be monitored is at home and the watcher is a visiting medical worker, on the other hand, the vital data transmitted from the wearable terminal 20 is transmitted by the dedicated transceiver 40 to the management server 10 over the communication network 2. Upon receiving the vital data, the management server 10 transmits the vital data to a watcher terminal 50c associated with the wearable terminal 20. Data processing in the watcher terminal 50c that has received the vital data is the same as that described above for the watcher terminal 50a or 50b.

Now, the data processing performed by the vital change detection means included in the first vital data processing means 51 will be described while taking examples. The vital change detection means detects physical events including disease onset and physical condition changes through a process in which changes in vital data are detected and compared with a predetermined threshold or a process in which changes in vital data in a plurality of measurement items are combined together. Here, the “physical events including disease onset and physical condition changes” to be detected refer to occurrence of abnormalities in a body (transition to states different from before) exemplified by awakening during sleep (mid-sleep awakening), onset of atrial fibrillation, anemia or heat stroke and associated syncope, fever, a sign of a pulse stop, and a pulse stop.

### Awakening During Sleep (Mid-Sleep Awakening) and Getting Out of Bed

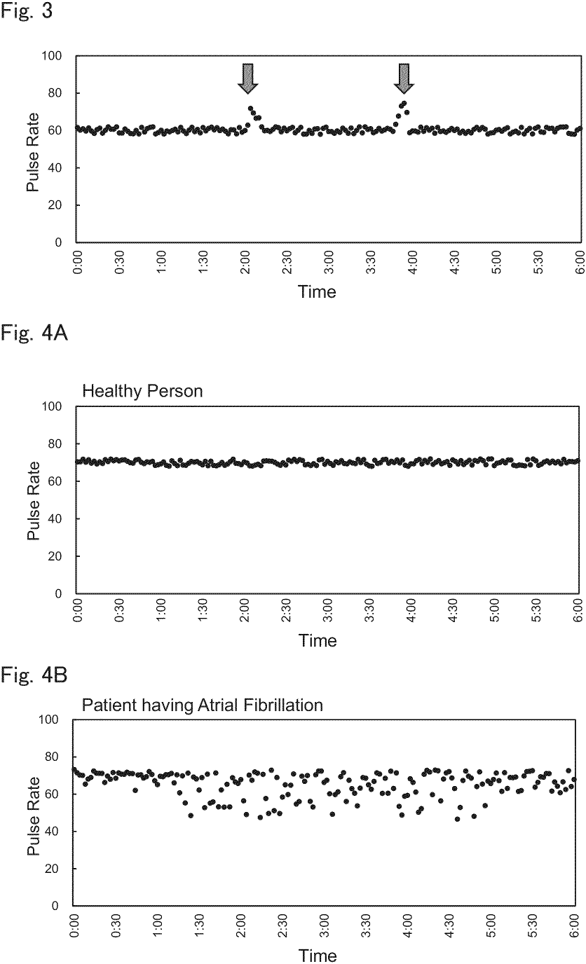

FIG. 3 is a graph where a pulse rate and skin temperature during sleep are plotted against time. The pulse rate during sleep is stable at low values, but temporarily increases upon a mid-sleep awakening (indicated by an arrow in the figure). By comparing the pulse rate in the vital data continuously obtained by the wearable terminal 20 with a pulse rate a certain period of time ago (e.g., three to five minutes ago), therefore, it can be determined that there has been a mid-sleep awakening (detection of a physical event) if a difference (the amount of change) remains larger than or equal to a certain threshold (e.g., 15 to 20) for a certain period of time (e.g., two to three minutes) or longer. If a further increase in the pulse rate is detected and the number of steps taken is measured thereafter, it can be detected, on the basis of these, that the person to be monitored has gotten out of bed and begun to move.

When a person to be monitored, such as an elderly person, awakes at night and wanders or goes to a bathroom alone, the person might fall and break a bone or injure himself/herself. If the person breaks a bone, for example, a level of care required increases, and burdens on both the person and a medical worker increase. In order to avoid such an event, medical workers make frequent nighttime rounds at nursing homes and hospitals, which places a heavy burden on the medical workers. There is also a system that monitors movement of a person to be monitored by installing a camera that captures an image of scene around a bed of the person and processing the captured image. In this case, however, the system detects the movement only after the person awakes and gets out of bed. By a time a medical worker is notified on the basis of the detection, therefore, the person might not be there, or has fallen down at a place where he/she has gone. In order to avoid such problems, some facilities administer sleeping pills to keep persons to be monitored from waking up at night, but there is concern that this might increase a physical burden on the persons.

In view of these problems, the watcher terminal 50 is notified of occurrence of an abnormality as soon as a person to be monitored wakes up, that is, before the person gets out of bed, in the monitoring assistance system 1 according to the present embodiment. This makes it possible for a watcher to respond quickly by, for example, calling out, through the camera apparatus 30, to the person who has woken up before he/she starts to move, thereby preventing the person from wandering alone.

### Onset of Atrial Fibrillation

FIGS. 4A and 4B are graphs where resting pulse rates and skin temperature of a healthy person and a patient with atrial fibrillation, respectively, plotted against time. As can be seen from FIG. 4A, the pulse rate of the healthy person at rest is stable at low values, whereas, as illustrated in FIG. 4B, the pulse rate of the patient with atrial fibrillation does not stabilize even at rest and greatly varies in some periods of time. By detecting a difference between a maximum value and a minimum value of a pulse rate in vital data at rest, such as during sleep, in a certain period of time (e.g., 3 to 5 minutes), therefore, it can be determined that there has been onset of atrial fibrillation (detection of a physical event) if the difference remains larger than or equal to a certain threshold (e.g., 3 to 7) for a certain period of time (e.g., 10 to 30 minutes) or longer. In addition, when an atrial fibrillation occurs, a cycle of a pulse wave is more irregular than usual. Onset of atrial fibrillation, therefore, can be detected by comparing a standard deviation of PPIs with a certain threshold or by comparing HRV with a certain threshold.

Atrial fibrillation is an arrhythmia caused by abnormal electrical excitation in atria. Because the atria contract irregularly as if in spasm, a pulse rate irregularly varies. Blood is not pumped normally from within the atria, and blood clots tend to be formed. If an atrial fibrillation occurs frequently, cerebral infarction and dementia can be caused. Atrial fibrillation ca thus be a cause of serious diseases, but because it is painless and imperceptible, there is concern that detection thereof might be delayed. In addition, atrial fibrillation often remains undetected even by electrocardiograms occasionally taken at hospitals, because it is not known when it will manifest. It is said that continuous week-long electrocardiography is necessary to detect onset of atrial fibrillation with electrocardiograms, but such a large-scale examination is difficult to perform.

In view of these conventional problems, vital data constantly measured by the wearable terminal 20 in the monitoring assistance system 1 according to the present embodiment includes a pulse rate and PPIs (or HRV). Even onset of atrial fibrillation, which can occur at any time, therefore, can be detected with a high probability.

### Anemia or Heat Stroke and Associated Syncope

Blood pressure and a pulse rate rise and fall in tandem with each other when normal. When physical exercise is started, for example, blood pressure rises and a pulse rate also rises, and when physical exercise is stopped, blood pressure decreases and returns to a normal value, and the pulse rate also decreases and returns to a normal value. In contrast, when anemia occurs or syncope occurs as a result of anemia, blood pressure suddenly decreases while a pulse rate increases. A difference between values of blood pressure and a pulse rate in vital data at the same point in time, therefore, may be detected, and if the difference is larger than a certain threshold, that is, if the blood pressure and the pulse rate do not change in tandem with each other, it can be determined that a person is prone to anemia or associated syncope (detection of a physical event). If the pulse rate increases in discrepancy from the blood pressure and skin temperature increases above a certain threshold, it can be determined that a person is prone to heat stroke and associated syncope (detection of a physical event).

### Fever

When the number of steps taken (activity level) in vital data is close to zero (within a range of 0 to “0 + a certain threshold”), a person is considered to be at rest. If, in this state, it is detected, as a result of detection of changes in the vital data, that both a pulse rate and skin temperature have increased above their respective certain thresholds, it can be determined that the increase in the pulse rate and body temperature is not a healthy one due to physical exercise but a fever caused by a disease (detection of a physical event).

### Sign of Pulse Stop and Pulse Stop

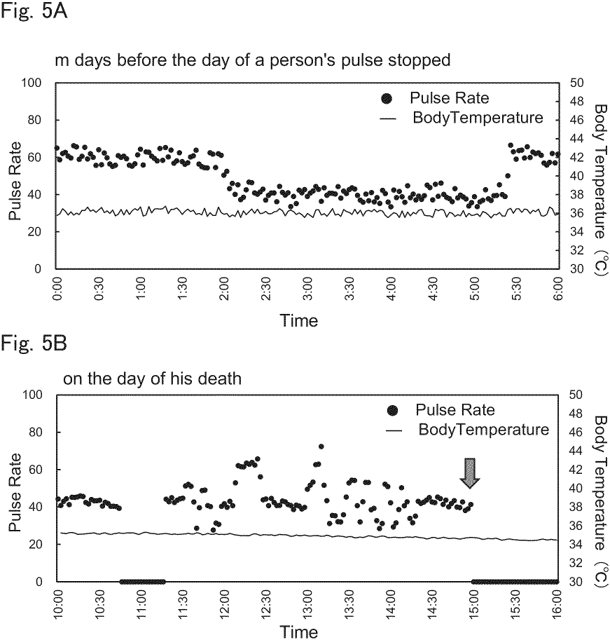

It has been conventionally thought that when a person dies quietly in a bedridden state without taking life-support measures, a pulse rate is substantially constant as during sleep and gradually decreases toward an end of life. Contrary to this conventional common wisdom of those skilled in the art, the present inventors have found, as illustrated in FIG. 5A, that, even in a bedridden state, a pulse rate greatly varies in a block-like manner one to two weeks before a pulse stops (a period in which the pulse rate continues to be high and a period in which the pulse rate continues to be low repeat irregularly). On a day when death is declared, the pulse rate, which has become low, repeats irregular fluctuation at short intervals and the skin temperature decreases until the pulse stops as illustrated in FIG. 5B (indicated by an arrow in the figure).

With the monitoring assistance system 1 according to the present embodiment, therefore, if it is detected that a pulse rate has remained lower than or equal to a certain threshold (e.g., 39) for a certain period of time (e.g., 10 to 15 minutes) or longer, it is determined that a pulse will stop before long (a sign of a pulse stop) and an alert signal is generated. The watcher terminal 50 then notifies of occurrence of an abnormality. As a result, a medical worker and family members contacted by the medical worker can, without delay, care for an end of life of a person to be monitored.

### Distinction Between Pulse Stop and Non-Wearing State

When a pulse of a person to be monitored stops, data regarding a pulse rate in vital data is no longer detected. It seems that a pulse stop can be detected on the basis of this, but the pulse rate is not detected either when the person has removed the wearable terminal 20 for bathing or other purposes. When pulse rate data is not being detected, it is necessary to distinguish whether a cause of the undetected pulse rate data is a pulse stop or the non-wearing state of the wearable terminal 20, because a pulse stop might occur without any signs being detected.

When vital data to be obtained by the wearable terminal 20 is not being detected, that is, in a following state, therefore, the vital change detection means determines that the wearable terminal 20 is not being worn.
Number of steps taken (activity level): Not detected
Pulse rate: Not detected
Blood pressure: Not detected
Blood oxygen level: Not detected
Body temperature: Not detected

Here, the body temperature data is calculated on the basis of body surface temperature and infrared radiation temperature as described above. In the non-wearing state, the sensor for detecting body surface temperature detects air temperature, but the sensor for detecting infrared radiation temperature does not detect temperature. The body temperature data, therefore, is not detected.

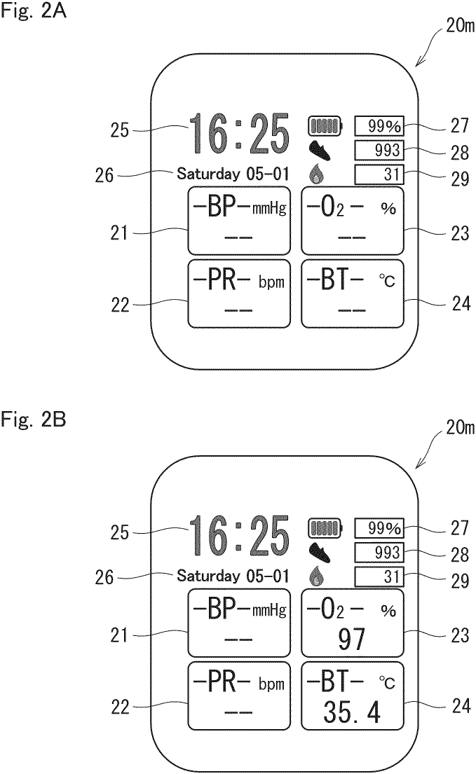

When a three-axis acceleration sensor for measuring the number of steps taken (activity level), a sensor for receiving reflection of green light in order to measure the pulse rate and the blood pressure, a sensor for receiving reflection of red light and infrared light in order to measure the blood oxygen level, and a sensor for detecting infrared radiation in order to measure the body temperature do not detect anything for a predetermined period of time, the wearable terminal 20 displays a non-detection indication “--” in a blood pressure display area 21, a pulse rate display area 22, a blood oxygen level display area 23, and a body temperature display area 24 of a monitor 20m, as illustrated in FIG. 2A, and sends zero as each piece of vital data. In addition to the above indication, the monitor 20m of the wearable terminal 20 is also provided with a time display area 25, a date display area 26, a battery level display area 27, a number of steps display area 28, and a calorie consumption display area 29. Calory consumption is calculated on the basis of the number of steps taken.

If the vital change detection means detects that the wearable terminal 20 has not detected the pulse rate and the blood pressure but has detected the blood oxygen level and the body temperature for a predetermined period of time (e.g., 3 to 40 minutes), on the other hand, the vital change detection means determines that a pulse has stopped, generates an alert signal, and causes the watcher terminal 50 to notify of occurrence of an abnormality. This process is based on a fact that when the pulse stops, the pulse rate and blood pressure data are no longer detected but the blood oxygen level and the body temperature continue to be detected. That is, when the pulse stops, a function of carrying oxygen through blood vessels stops, but because consumption of oxygen by a human body also stops, oxygen remains in blood and the blood oxygen level is detected continuously. In addition, because body temperature decreases after death by about 1° C. every 10 hours in an ordinary environment, the body temperature hardly changes in a period of time assumed as time taken to generate the alert signal.

In order to confirm this, chicken meat that had been sold in a supermarket store for human consumption within a day of being slaughtered was warmed to approximately 36° C., which is close to body temperature of a human body. A few hours after the warming was stopped, a sensing surface of the wearable terminal 20 was brought into contact with the chicken meat. As indicated by the monitor 20 m illustrated in FIG. 2B, a pulse rate and blood pressure were not detected, but a blood oxygen level was detected as 97% and body temperature was detected as 35.4° C. That is, these indications correspond to ones on the monitor at least several hours after a pulse stops.

The monitoring assistance system 1 can thus detect, when pulse rate data is not being detected, a pulse stop by clearly distinguishing the pulse stop from the non-wearing state of the wearable terminal 20, and causes the watcher terminal 50 to notify of occurrence of an abnormality. As a result, a situation where a person to be monitored dies unnoticed can be avoided even when the person is geographically distant from a visiting medical worker or a family member who is a watcher and does not see the watcher every day, such as when the person lives on his/her own. In addition, in the past, sufficient nighttime rounds could not be made due to a lack of manpower even in care facilities and hospitals, and a patient was sometimes found dead several hours after the death. By using the monitoring assistance system 1, however, such an unfortunate situation can be avoided.

A pulse stop is one of conditions under which a doctor declares death. A doctor declares death when heartbeat stops and pupils become dilated in addition to a pulse stop.

### Detection of Non-Wearing State Based on Skin Impedance

In the present embodiment, as described above, when vital data including body temperature data is not being detected, the wearable terminal 20 is determined to be in the non-wearing state. This is based on an assumption that in the non-wearing state, the sensor for detecting body surface temperature detects air temperature whereas the sensor for detecting infrared radiation does not detect temperature, and the body temperature data calculated on the basis of body surface temperature and infrared radiation temperature is not detected. If an object of which infrared radiation temperature is close to body temperature should exist near the wearable terminal 20 in the non-wearing state, however, the sensor for detecting infrared radiation might measure the infrared radiation temperature, and body temperature data might be detected. In order to detect the non-wearing state more reliably, therefore, measurement of skin impedance may be added.

A sensor for measuring skin impedance includes a pair of electrodes on a surface of the wearable terminal 20 that contacts skin (body surface) when the wearable terminal 20 is being worn, and is a sensor that applies a weak high-frequency current to the skin to detect a voltage or a sensor that applies a high-frequency voltage to the skin to detect a current. The skin impedance is a voltage divided by a current and, when expressed as a complex number, a real part is a resistance, and an imaginary part is a phase difference between the current and the voltage. When the wearable terminal 20 is being worn, the skin acts as a large electrical resistance, and the phase difference between the current and the voltage is small. When the wearable terminal 20 is not worn, on the other hand, impedance is almost infinite, and the phase difference between the current and the voltage is very large. The vital change detection means, therefore, can refer to the phase difference between the current and the voltage obtained on the basis of measurement of skin impedance when pulse rate data is not being detected and body temperature data is being detected, for example, and if an absolute value of the phase difference is larger than a predetermined threshold, determine that the non-wearing state has been established. In this case, the detected body temperature data does not reflect the wearer’s body temperature, and monitor indications are as described with reference to FIG. 2A.

Mental data may be included in vital data to be obtained when the wearable terminal 20 includes a sensor for measuring skin impedance in order to reliably detect the non-wearing state. When a person is mentally excited or under strong stress, a sympathetic nervous system activates sweat glands and promotes sweating. Sweating causes the skin impedance to decrease because electrical conductivity of the skin increases. By continuously measuring the skin impedance, therefore, average values at rest and under normal conditions can be identified, and by detecting a time when the skin impedance falls below a predetermined range, it is possible to detect, as a physical event, that a person has reached a state of excitement or is under strong stress.

A case has been described where physical events are detected in almost real-time through the immediate data processing performed by the first vital data processing means 51. In the monitoring assistance system 1, the second vital data processing means 13 also performs data processing, which is performed using the target quantitative information 12d accumulated in the database 12 over a certain period of time. The second vital data processing means 13 performs the data processing on new vital data using the analysis result information 12e created on the basis of past vital data and estimates or predicts physical events for a person to be monitored who has shown the new vital data. The estimation and prediction of physical events are exemplified as prediction of diseases that will develop in the near future and prediction of a pulse stop in the near future, but since the estimation and prediction have already been proposed in another application, detailed description thereof is omitted.

Although the present invention has been described with reference to a preferred embodiment, the present invention is not limited to the above embodiment and may be improved or modified in various ways without departing from the aspect of the present invention.

Although the wearable terminal 20 is worn on a wrist like a wristwatch in the above embodiment, for example, the wearable terminal 20 is not limited to this. The wearable terminal 20 may be worn on another part of a human body, such as an ankle or an upper arm, instead.

In addition, although the first vital data processing means 51 is a functional component of the watcher terminal 50 in the above embodiment, the first vital data processing means 51 may be a functional component of the management server 10, instead. In this case, vital data transmitted from the wearable terminal 20 is transmitted to the management server 10 through the dedicated transceiver 40, and the management server 10 detects a physical event on the basis of changes in the vital data. If the management server 10 detects a physical event, the management server 10 transmits an alert signal to the watcher terminal 50, and the watcher terminals 50 notifies of occurrence of an abnormality.

What is claimed is:

1. A monitoring assistance system comprising:
a wearable terminal that is worn by a person to be monitored and that continuously obtains vital data;
a watcher terminal associated with the wearable terminal; and
a management server communicably connected to at least one of the watcher terminal or the wearable terminal,
wherein at least one of the watcher terminal or the management server includes first vital data processing means for receiving the vital data to which an identification code of the wearable terminal is attached and performing data processing on the vital data,
wherein the vital data includes pulse rate data and blood oxygen level data, and
wherein the first vital data processing means determines, in a case where the pulse rate data is not being detected and the blood oxygen level data is not being detected, that a non-wearing state, where the wearable terminal is removed from the person to be monitored, has been established, and, in a case where the pulse rate data is not being detected and the blood oxygen level data is being detected, that a pulse of the person to be monitored has stopped, generates an alert signal, and causes the watcher terminal to notify.

2. The monitoring assistance system according to claim 1,
wherein the vital data includes body temperature data,
wherein the body temperature data is deep body temperature data calculated on a basis of body surface temperature and infrared radiation temperature measured by the wearable terminal, and
wherein, in a case where the pulse rate data is not being detected and the blood oxygen level data and the body temperature data are being detected, the first vital data processing means determines that the pulse of the person to be monitored has stopped.

3. A monitoring assistance method in a monitoring assistance system including
a wearable terminal that is worn by a person to be monitored and that continuously obtains vital data,
a watcher terminal associated with the wearable terminal, and
a management server communicably connected to at least one of the watcher terminal or the wearable terminal,
wherein the vital data to which an identification code of the wearable terminal is attached is processed, the monitoring assistance method comprising:
including pulse rate data and blood oxygen level data in the vital data;
determining, in a case where the pulse rate data is not being detected and the blood oxygen level data is not being detected, that a non-wearing state, where the wearable terminal is removed from the person to be monitored, has been established; and
determining, in a case where the pulse rate data is not being detected and the blood oxygen level data is being detected, that a pulse of the person to be monitored has stopped.
